(19) United States
(12) Patent Application Publication
(10) Pub. No.: US 2024/0041312 A1
(43) Pub. Date: Feb. 8, 2024

(54) ENDOSCOPIC PATCH DELIVERY

(71) Applicants: Boston Scientific Scimed, Inc., Maple Grove, MN (US); Boston Scientific Medical Device Limited, Galway (IE)

(72) Inventors: Hitendra PUROHIT, VADODARA (IN); Deepak Kumar SHARMA, Muzaffarnagar (IN); Lauren Sfakis LYDECKER, Millbury, MA (US); Paul SMITH, Smithfield, RI (US); Madhurima DEBNATH, Naihati (IN); Aditya DHANOTIYA, Indore (IN)

(73) Assignees: Boston Scientific Scimed, Inc., Maple Grove, MN (US); Boston Scientific Medical Device Limited, Galway (IE)

(21) Appl. No.: 18/360,897

(22) Filed: Jul. 28, 2023

Related U.S. Application Data
(60) Provisional application No. 63/394,651, filed on Aug. 3, 2022.

(51) Int. Cl. A61B 1/31; A61B 1/005; A61B 1/00; A61B 17/00

(52) U.S. Cl. CPC A61B 1/31; A61B 1/0052; A61B 1/00082; A61B 17/00234; A61B 2017/00269

(57) ABSTRACT

A medical device that includes: a first handle coupled to a first sheath; a second handle coupled to a second sheath, wherein at least a portion of the first sheath is disposed within a lumen of the second sheath; and a patch. In a first configuration, the first handle and the second handle may be separated by a first distance, and the patch may be disposed between a distal portion of the first sheath and a distal portion of the second sheath. In a second configuration, the first handle and the second handle may contact one another or may be separated by a second distance, wherein the second distance is smaller than the first distance, and wherein a distalmost end of the second sheath is proximal of a proximal end of the patch.

### CROSS-REFERENCE TO RELATED APPLICATIONS

This application claims the benefit of priority of U.S. Provisional Patent Application No. 63/394,651, filed Aug. 3, 2022, the entirety of which is incorporated herein by reference.

### TECHNICAL FIELD

The disclosure relates generally to medical devices and related methods of preparation and use thereof. More specifically, the disclosure includes devices useful in endoscopic medical procedures, such as applying a patch to tissue for resection. More particularly, embodiments of the disclosure relate to minimally invasive devices and methods for full thickness endoscopic mucosal resection. The disclosure also relates to methods of using such devices.

### BACKGROUND

Colorectal cancer is the second leading cause of cancer death in the U.S. Current treatment options for removal of colorectal lesions (including cancerous and pre-cancerous lesions) include endoscopic treatment or surgery. Examples of endoscopic treatment include endoscopic mucosal resection (“EMR”) or endoscopic full thickness resection (“EFTR”). EMR may be utilized for resection of superficial lesions (e.g., lesions confined to the mucosa or submucosa) of the colorectum. EFTR may be utilized to treat lesions of the colorectum that extend into deeper layers of colorectal tissue. However, EFTR has traditionally been associated with risks of intrabdominal hypertension, insufflation, and/or seeding of cancer cells. Because endoscopic resection, done in an outpatient setting, has the potential to dramatically reduce hospital stay as well as morbidity and mortality associated with surgical resection, there is an unmet need for endoscopic tissue dissection of malignant and pre-malignant lesions, including lesions extending deeper than those lesions treatable with EMR.

### SUMMARY

The present disclosure includes medical devices and systems comprising a biocompatible patch and methods of use thereof, e.g., methods of delivering a patch to a target site of a patient to safely resect target tissue.

In an example, a medical device may comprise: a first handle coupled to a first sheath; and a second handle coupled to a second sheath. At least a portion of the first sheath may be disposed within a lumen of the second sheath. The medical device also may comprise a patch. In a first configuration, the first handle and the second handle may be separated by a first distance, and the patch may be disposed between a distal portion of the first sheath and a distal portion of the second sheath. In a second configuration, the first handle and the second handle may be contacting one another or may be separated by a second distance, wherein the second distance is smaller than the first distance, and wherein a distalmost end of the second sheath is proximal of a proximal end of the patch.

Any of the examples described herein may have any of the following features in any combination. The first handle of the medical device may comprise a proximal opening configured to receive a distal portion of a handle of an endoscope. The first handle of the medical device may comprise a feature configured to accommodate a feature of an endoscope. The patch of the medical device may comprise at least one pull line. At least one pull line may be arranged within a perimeter of the patch and conform to a shape of the perimeter of the patch. Each of the at least one pull lines may terminate in two free ends extending from a same edge of the patch. The first handle of the medical device may comprise an actuator configured to move at least one pull line in a proximal direction. A distal portion of the first sheath may include a balloon coupled to an external surface of the first sheath. In the first configuration, the balloon may be disposed between the first sheath and the patch. In a third configuration, the balloon may be inflated and the patch may be disposed on the inflated balloon. In a fourth configuration, the balloon and the patch may be separated. The patch may include an adhesive on a side of the patch facing away from the first sheath. The patch may be exposed to a liquid via a fluid channel. The medical device may be removably coupled to the endoscope by means of one or more fasteners, adhesives, or a friction fit. The first sheath may comprise a lumen to receive a shaft of an endoscope.

In an additional or alternative configuration, a medical device may comprise: a first sheath and a second sheath. The first sheath may be disposed within a lumen of the second sheath. The medical device also may comprise a balloon and a patch. In at least one configuration of the medical device, at least a portion of the patch may be disposed between the second sheath and at least a portion of the balloon, and at least a portion of the balloon may be disposed between at least a portion of the patch and the first sheath. The patch may further comprise at least one pull line arranged within a perimeter of the patch and conform to a shape of the perimeter of the patch. The at least one configuration may be a first configuration and, in a second configuration, the second sheath may be retracted relative to the first sheath and the balloon may be inflated.

In an additional or alternative example, a medical method may comprise: deploying a patch over a lesion within a body lumen; pulling a pull line of the patch in a proximal direction, such that the patch surrounds at least a portion of the lesion and a first tissue portion on a first side of the lesion and a second tissue portion on a second side of the lesion are pulled together; cutting the patch and the lesion contained therein; and removing the patch and the lesion from the body lumen. The method may further include: before deploying the patch, introducing a distal portion of a medical device into a body lumen; and after introducing the distal portion of the medical device into the body lumen, moving a second handle of the medical device proximally relative to a first handle of the medical device, thereby moving a second sheath of the medical device proximally relative to a first sheath of the medical device and exposing the patch at the distal portion of the medical device.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts a perspective view of a medical device assembly, according to aspects of this disclosure.

FIG. 2 depicts an exemplary patch, according to aspects of this disclosure.

FIGS. 3A-3G depict the device of FIG. 1 deploying the patch of FIG. 2, according to aspects of this disclosure.

FIGS. 4A and 4B depict an exemplary device in an open configuration (FIG. 4A) and in a closed configuration (FIG. 4B), according to aspects of this disclosure.

FIGS. 5A and 5B depict an alternative device in an open configuration (FIG. 5A) and in a closed configuration (FIG. 5B), according to aspects of this disclosure.

FIG. 6 depicts an alternative device, according to aspects of this disclosure.

### DETAILED DESCRIPTION

Reference is now made in detail to examples of the present disclosure, aspects of which are illustrated in the accompanying drawings. Wherever possible, the same reference numbers will be used throughout the drawings to refer to the same or like parts.

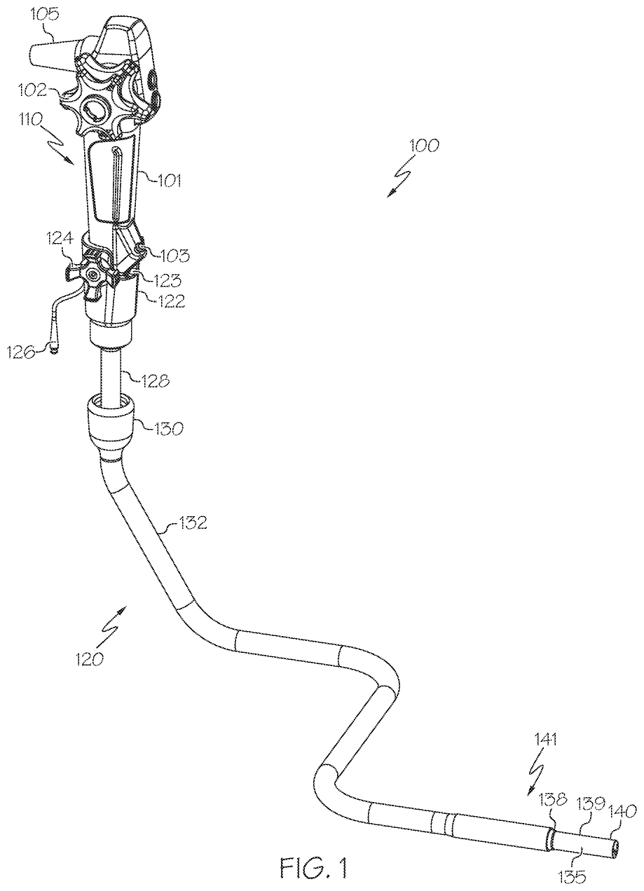

FIG. 1 depicts a perspective view of a medical device assembly 100, including a scope 110 and a medical device 120. Although the term scope may be used herein, it will be appreciated that this term encompasses a variety of devices, including, but not limited to, endoscopes, duodenoscopes, colonoscopes, ureteroscopes, bronchoscopes, or laparoscopes, and other devices, including, but not limited to sheaths, catheters, or any other suitable delivery device or medical device may be used in connection with the devices of this disclosure. Scope 110 comprises a handle 101 (which may remain outside of a subject's body) and an insertion sheath 139 (i.e., a shaft) (which may be inserted or introduced into a body lumen of the subject).

Handle 101 may include one or more control mechanisms 102. Control mechanisms 102 may enable deflection/steering of a steerable section 135 of a distal portion of insertion sheath 139 to allow an operator to navigate the insertion sheath 139 through tortuous anatomy and/or towards a site of interest. Handle 101 may include a port 103, which may be in fluid communication with one or more lumens (e.g., working channels) of insertion sheath 139. Scope 110 may also include an umbilicus 105 extending from handle 101 for purposes of connecting scope 110/handle 101 to sources of, for example, air, water, suction, power, etc., as well as to image processing and/or viewing equipment (not shown).

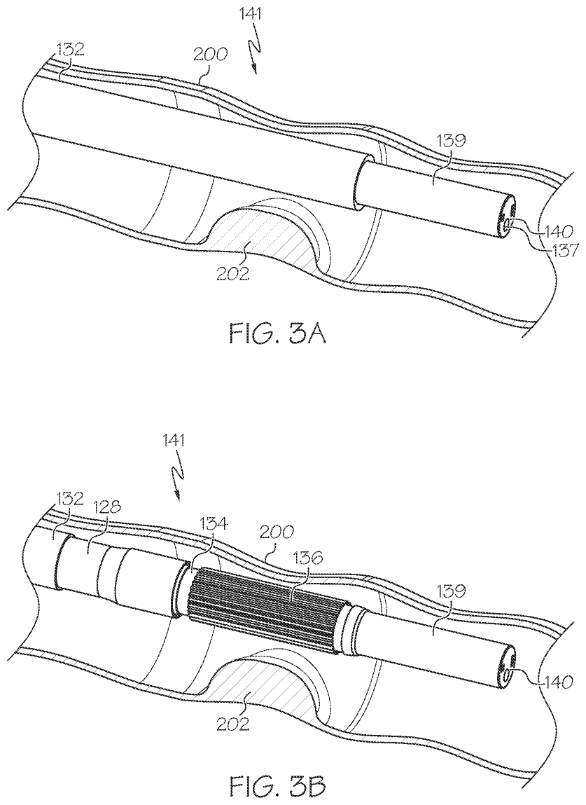

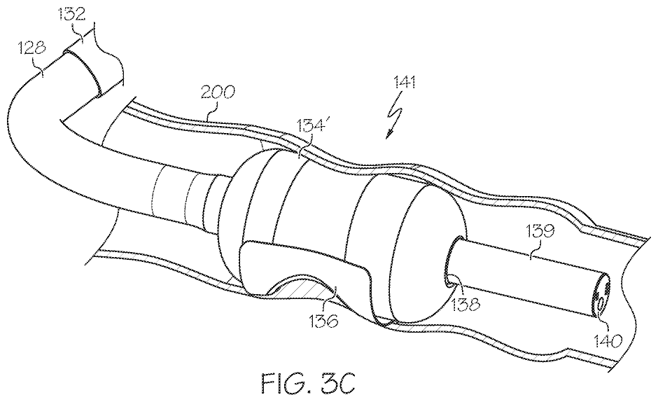

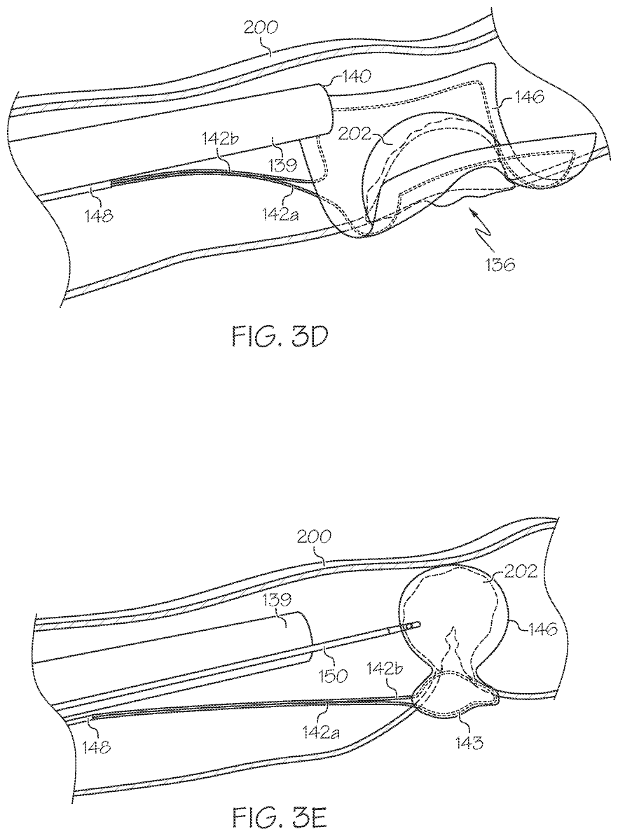

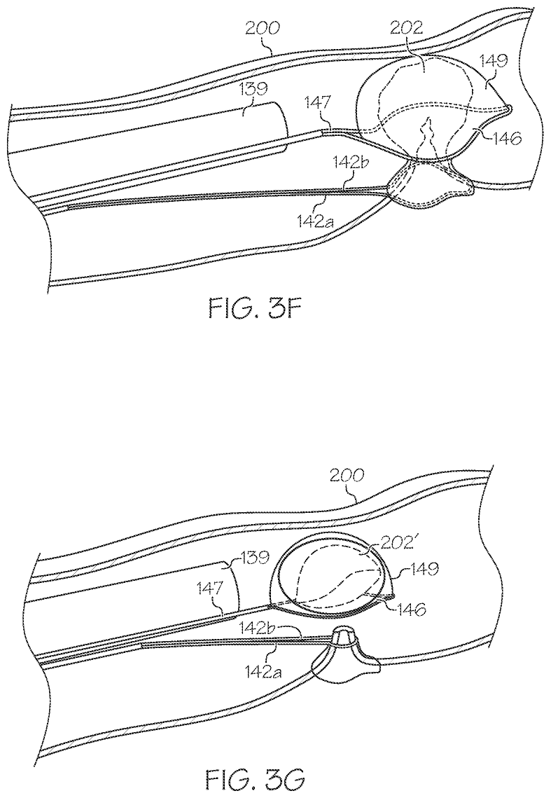

Insertion sheath 139 extends from a distal portion of handle 101 to a distal end 140 of scope 110. Insertion sheath 139 may include one or more lumens (e.g., working channels, air/water lumens, wire/cable lumens, etc.). For example, a working channel of insertion sheath 139 may terminate in a distal opening 137 at distal end 140 (shown in FIG. 3A). As mentioned above, the distal portion of insertion sheath 139 may include a steerable section 135. Steerable section 135 may include, for example, an articulation joint. Insertion sheath 139 may be sufficiently flexible to maneuver through different anatomical structures, including portions of the gastrointestinal system. Insertion sheath 139 and steerable section 135 may include a variety of structures that are known or may become known in the art. For example, distal end 140 of scope 110 may include a distal tip having an imaging device (e.g. a camera) and a lighting source (e.g. an LED or optical fiber) (not shown in FIG. 1).

Medical device 120 may include a primary, or first, handle 122 and a secondary, or second, handle 130. Primary handle 122 may include a fluid supply connector 126 and one or more actuators 124 (e.g. knobs), to be described in further detail below. Primary handle 122 is approximately cylindrical in shape and wraps around a distal portion of handle 101. Primary handle 122 may feature various cutouts or contours to accommodate one or more features of handle 101 of scope 110. For example, primary handle 122 may include a cutout 123 to receive a portion of port 103 of handle 101.

An internal sheath 128 extends from a distal portion of primary handle 122. Internal sheath 128 may extend about insertion sheath 139 (e.g., insertion sheath 139 may be disposed within a lumen of internal sheath 128). An external sheath 132 extends from a distal portion of secondary handle 130. Internal sheath 128 extends through a center opening of secondary handle 130 and through a central lumen of external sheath 132 to a distal end 138 of medical device 120. External sheath 132 is shorter in overall length as compared to internal sheath 128. Internal sheath 128 and external sheath 132 may be coated with or made from a lubricious material to assist with loading or unloading medical device 120 onto scope 110 (as discussed below) and/or to assist with the movement of external sheath 132 relative to internal sheath 128. External sheath 132 and internal sheath 128 may be comprised of flexible biocompatible materials to not impede or otherwise restrict the flexibility of insertion sheath 139 of scope 110. Portions of external sheath 132 and internal sheath 128 may comprise different materials or different thicknesses of the same material to create variability in flexibility along the length of the sheaths. Additionally or alternatively, external sheath 132 may be comprised of one material and internal sheath 128 may be comprised of a second material, different from external sheath 132.

External sheath 132 and internal sheath 128 may further comprise additional lumens to assist with the delivery and/or removal of additional medical devices or fluids (i.e., air, water). For example, external sheath 132 or internal sheath 128 may comprise one or more additional lumens running proximally to distally along a length of the respective sheath to facilitate the use of one or more additional medical devices, such as a wires, forceps, baskets, nets, guidewires, or any other device commonly used in the art. Additionally or alternatively, one or more lumens of internal sheath 128 may be coupled to fluid supply connector 126 and may be used to facilitate the expansion or contraction of a balloon 134, described in further detail below. A separate lumen (not shown) or a shared lumen may be used to facilitate the connection between pull lines 142a, 142b (discussed in detail below) of a patch 136 (not visible in FIGS. 1 and 3A but shown in FIG. 3B) to actuators 124 of primary handle 122.

In a first configuration, as shown in FIGS. 1 and 3A, primary handle 122 and secondary handle 130 are separated by a first distance, or not touching. More specifically, secondary handle 130 is positioned distally along internal sheath 128 as compared to primary handle 122, such that a gap is formed between a distal end of primary handle 122 and a proximal end of secondary handle 130. In this configuration (also shown in FIG. 3A), a deflated balloon 134 and patch 136 (not visible in FIGS. 1 and 3A but shown in FIG. 3B) may be positioned between an external surface of internal sheath 128 and an internal surface of external sheath 132 towards a distal end 138 of medical device 120. This arrangement is shown in more detail in FIGS. 3A-3C. For example, as shown in more detail in FIG. 3B, patch 136 may be positioned about deflated balloon 134 (radially outside of balloon 134 as compared to a central longitudinal axis of internal sheath 128). Moreover, in the first configuration, a distalmost end of internal sheath 128 may align with a distalmost end of external sheath 132 at distal end 138 of medical device 120. For at least this reason, a distalmost end of internal sheath 128 is not visible in FIG. 3A or near distal end 138 of FIG. 1.

In the first configuration, the distalmost ends of internal sheath 128 and external sheath 132 terminate proximally to steerable or articulation section 135 of scope 110. In alternative arrangements of medical device 120 in the first configuration, a distal portion 141 of medical device 120 (e.g., internal sheath 128 and/or external sheath 132 in the first configuration of medical device 120) may terminate distally to steerable or articulation section 135 of scope 110, near distal end 140. In such an embodiment, patch 136 and balloon 134 may still be positioned proximally to steerable or articulation section 135 of scope 110 to not interfere with the articulation or steerability of scope 110.

FIG. 3B shows distal portion 141 of medical device 120 in a second configuration. In the second configuration, secondary handle 130 is pulled proximally (i.e., upwards in the direction of FIG. 1) such that the primary handle 122 and secondary handle 130 are closer to one another and/or in contact with one another (not shown in FIG. 1). For example, the primary handle 122 and second handle 130 may be separated by a second distance. The second distance may be smaller than the first distance, discussed above with reference to the first configuration. In some configurations, primary handle 122 may be nested within a proximal opening of secondary handle 130. In the second configuration, as secondary handle 130 is pulled upwards, external sheath 132 is also pulled proximally while internal sheath 128 remains stationary, thus exposing deflated balloon 134 and patch 136.

As shown in FIG. 3C and described in further detail below, in a third configuration of medical device assembly 100, balloon 134 may then be inflated using fluid supplied through fluid supply connector 126 shown in FIG. 1. A lumen or tube (not shown) may extend the length of internal sheath 128 and be in fluid connection with balloon 134 in order to inflate balloon 134. Alternatively, the lumen or tube in communication with balloon 134 and fluid supply connector 126 may extend through other portions of medical device 120. Fluid supply connector 126 may be coupled to an external source, such as a fluid source or an air source. Various materials can be used to inflate balloon 134, including, but not limited to saline, water, air, etc. Inflation of balloon 134 may, in turn, move patch 136 radially outward. Patch 136 may conform to a shape of balloon 134. It is in this configuration that patch 136 may be applied to a target tissue 202 (i.e., a lesion). For example, as discussed below, balloon 134 may press patch 136 against target tissue 202. Balloon 134 may be comprised of any material commonly known in the art and used for balloons, such as, for example, polyurethane, Pebax™, and/or silicone.

In alternative configurations, balloon 134 may be substituted with an expandable element, such as an expandable basket or a cage (not shown). The expandable basket or cage may be configured or biased in an open or expanded configuration. For example, when external sheath 132 is pulled proximally, the basket or cage automatically expands, much like a balloon being inflated. Such alternative expandable elements may have the same or similar effects on patch 136 as described above, facilitating application of patch 136 to target tissue 202.

Medical device 120 may be attached to a distal portion of handle 101 of scope 110 by back loading medical device 120 onto scope 110, much like an arm in a sleeve, such that the handle of scope 110 is nested within a proximal opening of medical device 120 (such as a proximal opening of primary handle 122) and the insertion sheath 139 of scope 110 is positioned within a lumen of an internal sheath 128 of medical device 120. Medical device 120 may be held in place on scope 110 by means of a friction fit, one or more fasteners, adhesives, or any other method commonly known in the art.

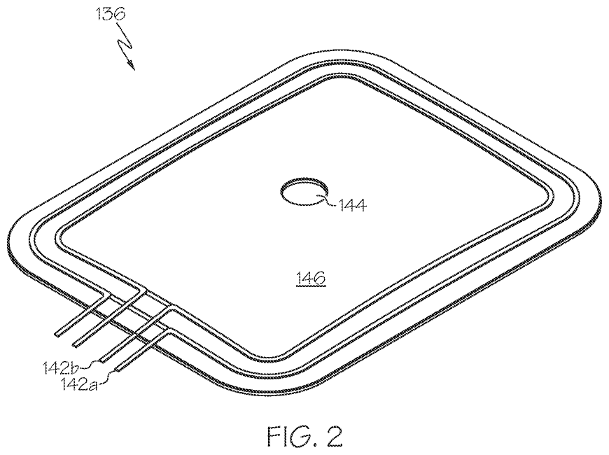

FIG. 2 depicts an exemplary patch 136. Patch 136 may be a biodegradable and/or biocompatible patch of any suitable shape and any suitable dimension, e.g. based on the nature of the target tissue site. Patch 136 may have any shape such as, e.g., approximately square, approximately rectangular, rounded square, rounded rectangle, ovate, circular, among other possible shapes. In some examples, the thickness of the patch may be on the order of millimeters, e.g. ranging from about 0.1 mm to 5.0 mm or, more specifically, from about 0.7 mm to about 2.0 mm. Patch 136 may be sufficiently sized to cover the target tissue with a margin for resection. Thus, patch 136 can come in many sizes to accomplish such a task. Moreover, patch 136 may be of any suitable color, including clear, and material, e.g., nettings, cloths, polysaccharides (chitosan, cellulose, starch, alginates, etc.) that may be further modified with synthetic biocompatible materials (pHEMA, PGA, PLA, PCA, PEG, etc.). Additionally, for example, patch 136 may be comprised of polypropylene, polyester, ePTFE, and/or silicone. Patch 136 may be adhered to the target tissue using materials commonly known in the art, such as, for example fibrin glue, hydrogel, and/or cyanoacrylate. Alternatively or additionally, patch 136 may be comprised of or dosed with agents to prevent the shedding of cells from the target tissue or to treat the target site.

One or more pull lines 142a, 142b may be arranged within a perimeter of patch 136 on one or more surfaces of patch 136 (e.g. an upper/top surface 146 depicted in FIG. 2) and mimic, or conform to, the overall shape of patch 136. Pull lines 142a, 142b may be positioned within one or more lumen(s) on or within patch 136 so as to permit moveability of pull lines 142a, 142b relative to patch 136 (e.g., relative to lumens of patch 136). In alternative configurations, pull lines 142a, 142b may be positioned on a lower/bottom surface (not shown in FIG. 2, and opposite top surface 146) or between layers of patch 136. Any combination is also permitted. For example, pull line 142a may be positioned between layers of patch 136 and pull line 142b may be positioned on an upper/top surface 146, or vice versa. Pull lines 142a, 142b may form approximately straight lines along the edges of patch 136 or may be oriented to form various curves or geometries. For example, as shown in FIG. 2, pull lines 142a, 142b may have a square or rounded square shape. Additionally or alternatively, pull lines 142a, 142b may form a circle on a square or rectangular patch 136.

The number of pull lines 142a, 142b is not limited to two, as there may be additional pull lines or fewer pull lines. Moreover, pull lines 142a, 142b need not mimic each other's geometry and may have any suitable shapes. For example, pull line 142a may form a circle and pull line 142b may form a square encompassing the circle. It may be understood that other geometries are also permitted. Pull lines 142a, 142b may be comprised of any suitable material, including metal, thread, a suture material, etc. Pull lines 142a, 142b may also be coated with any desirable biocompatible material to assist in reducing the amount of force required to pull the pull lines 142a, 142b. Pull lines 142a, 142b may be of any suitable length. For example, pull lines 142a, 142b may be sufficiently long enough to be coupled to actuator 124 of FIG. 1 (i.e., the pull line may extend the entire length of the internal sheath 128 of medical device 120). Alternatively, pull lines 142a, 142b may be cut short such that a secondary tool (not shown) may be used to pull on pull lines 142a, 142b while patch 136 is in position in a body lumen. In examples, pull lines 142a, 142b may extend through a pull line sheath 148 (FIGS. 3D-3F), which may, in turn, extend through a lumen of medical device 120 (e.g., a lumen of external sheath 132 or internal sheath 128) or through a space between external sheath 132 and internal sheath 128.

Pull lines 142a, 142b may have one or two free ends extending from patch 136. The free ends of pull lines 142a, 142b may extend from the same side of patch 136, as shown in FIG. 2, or may extend from opposing sides of patch 136. For example, the free ends of pull line 142a may extend from one side of patch 136, and the free of pull line 142b may extend from an opposite or opposing side of patch 136.

Patch 136 may further comprise a hole 144, extending through one or more layers of the patch material. Hole 144 may be configured and sized such that a grasper (not shown) can be passed through hole 144 to assist the user with manipulating or adjusting the target tissue contained within patch 136.

Patch 136 may be pliable and bendable. For example, patch 136 may be formed into a planar film, sheet, or disc capable of being folded, crimped, or otherwise manipulated into a temporary non-planar configuration within a confined space or when other forces act on patch 136. For example, patch 136 may be folded, curved, or crimped between internal sheath 128 and external sheath 132 of medical device 120 for suitable delivery via scope 110. Alternatively, patch 136 may be folded, curved, crimped, or otherwise manipulated to fit within a lumen of scope 110. However, once balloon 134 is inflated or once patch 136 is released from the balloon, patch 136 may recover its original planar, or other expanded, configuration.

Patch 136 may be substantially dry during delivery to a target site. In some examples herein, patch 136 may be moistened during delivery or shortly following release from a delivery instrument. For example, patch 136 may be exposed to water or another liquid via a fluid channel of medical device 120 or scope 110 and/or through contact with bodily fluids or humidity inside the body. The fluid channel (not shown) of medical device 120 may be in fluid connection with fluid supply connector 126 or a similar structure (i.e., a port). When exposed to moisture, patch 136 may absorb the fluid, causing patch 136 to swell or expand. For example, patch 136, when dry, may be substantially paper-like, and, when wet, may take on a more jelly-like consistency. According to some aspects of the present disclosure, moistening patch 136 may assist in its application to tissue.

In some examples herein, patch 136 comprises an adhesive, e.g., to assist in applying the patch to tissue and/or maintaining the patch in place after application, and/or to assist in delivery of patch 136 to the target site. The adhesive may be disposed on a surface of patch 136 opposite a surface of patch 136 having pull lines 142a, 142b. The adhesive may be disposed on a bottom surface (opposite top surface 146) of patch 136 in certain discrete locations or on the entirety of the bottom surface. Adhesives suitable for the present disclosure may be natural, e.g., comprising a natural polymer or derived from a natural polymer, or synthetic. Exemplary adhesives include, but are not limited to, gelatin (including, e.g., thrombin/gelatin), fibrin (e.g., fibrin glue), cyanoacrylate, polyethylene glycol (PEG), and albumin (including, e.g., albumin glutaraldehyde). The adhesive may be at least partially resorbable by a subject's body.

The choice of adhesive may be at least partially based on the desired adhesion strength, bioresorbable properties, and/or the nature of the target site to which patch 136 is being applied. For example, a relatively stronger adhesive may be desired for a patch 136 comprising an extracellular matrix (ECM) than for a patch 136 comprising chitosan, which has natural bioadhesive properties. Further, for example, a patch 136 intended for application to a relatively large wound, tumor, or defect site, may comprise adhesive to further assist with securing patch 136 to the tissue. Patch 136 according to the present disclosure may comprise adhesive applied to one area or two or more areas, e.g., opposing ends of patch 136. Alternatively or additionally, the adhesive may be applied within the perimeter of patch 136, leaving a center portion of patch 136 without adhesive. In some examples, patch 136 does not include an adhesive.

FIGS. 3A-3G depict medical device 120 of FIG. 1 deploying patch 136 of FIG. 2 within a body lumen. FIG. 3A depicts distal portion 141 of medical device 120 with medical device 120 in the first configuration, described in further detail above. For example, in FIG. 3A, distal end 140 of insertion sheath 139 of scope 110 is inserted, or introduced, into a body lumen 200. Distal end 140 of scope 110 is positioned past (distally of) a target tissue 202 (e.g. a tumor) such that a distal portion of external sheath 132 is positioned over (i.e., aligned with) the target tissue 202.

FIG. 3B depicts distal portion 141 of medical device 120 in the second configuration. As previously described, in FIG. 3B, secondary handle 130 (FIG. 1) has been pulled proximally (towards the operator or upwards in FIG. 1), while scope 110 remains in place (i.e., stationary). Proximal movement of secondary handle 130 results in retracting external sheath 132 proximally relative to internal sheath 128 and insertion sheath 139, in order to reveal a distal portion of internal sheath 128. The distal portion of internal sheath 128 has deflated balloon 134 and patch 136 mounted thereon. As discussed above, patch 136 may be mounted about balloon 134 (radially outward of balloon 134). In this state, patch 136 is crimped, folded, wrapped, or otherwise oriented so as to fit between external sheath 132 and balloon 134 (which is between external sheath 132 and internal sheath 128). The distal portion of medical device 120 is then positioned such that patch 136 and balloon 134 are aligned with the target tissue 202. Proximal and distal ends of balloon 134/patch 136 may extend beyond target tissue 202, such that there is a margin around the target tissue for sufficient resection. In examples, patch 136 and balloon 134 may be positioned so that they are approximately centered with respect to target tissue 202.

FIG. 3C depicts distal portion 141 of medical device 120 in the third configuration, described above. In FIG. 3C, balloon 134 is inflated, for example, via fluid supply connector 126 (FIG. 1). As discussed above, fluid supply connector 126 may be in fluid communication with balloon 134 via a lumen or channel, so as to enable the inflation and/or deflation of balloon 134. As discussed above, balloon 134 may be inflated by means of one or more fluids (i.e., saline or water) or gasses. Once inflated, balloon 134 may press patch 136 against target tissue 202. Patch 136 may adhere to the tissue. Balloon 134 may then be deflated to release patch 136, or balloon 134 may be pulled away from patch 136, leaving patch 136 in place, attached to target tissue 202, as shown in FIG. 3D.

FIG. 3D depicts a distal portion of insertion sheath 139 once patch 136 is positioned within lumen 200. In this configuration, balloon 134 (not shown in FIG. 3D but shown in FIGS. 3A-3C) may have been deflated and insertion sheath 139 may have been pulled proximally (i.e., towards the operator), allowing for a user to visualize patch 136 using the imaging device of scope 110 on distal end 140 of insertion sheath 139. Alternatively or additionally, portions of medical device 120 may be removed (e.g., external sheath 132 and/or internal sheath 128) from body lumen 200 and/or from scope 110. As shown in FIG. 3D, patch 136 may be secured to target tissue 202 (e.g., via adhesive of patch 136, discussed above).

As shown in FIG. 3E, once patch 136 is in position, one or both pull lines 142a, 142b may be pulled proximally (e.g., through pull line sheath 148) by rotating actuator 124 of FIG. 1 clockwise or counterclockwise. In some configurations, actuator 124 may include multiple actuators utilized to control each of pull lines 142a, 142b independently. For example, one of actuator 124 may be rotated to pull on pull line 142a, and a second actuator (not pictured) may be used to pull on pull line 142b. Alternatively or additionally, a secondary tool (e.g., forceps) can be used to pull on pull lines 142a, 142b. Because the patch is adhered to the tissue, tensioning or pulling one or more pull lines 142a, 142b brings opposing portions of the healthy section of the tissue wall together, cinching patch 136, and creating a pouch or a sack of tissue, including target tissue 202. This creates a shape similar to a polyp, enabling a user to use a secondary tool to more easily extract target tissue 202 within patch 136, similar to a polypectomy, as described below. This process reduces the chances of seeding while the tumor is being resected because the tumor is contained and confined within the patch.

A secondary tool 150 may be used to assist in positioning target tissue 202 within the pouch or sack created by patch 136. Secondary tool 150 may include forceps, guidewires, nets, graspers, or any other tool commonly known in the art. Secondary tool 150 may be inserted through a lumen of insertion sheath 139 of scope 110. In alternatives, secondary tool 150 may be inserted externally to scope 110, as shown. For example, secondary tool 150 may be an element of medical device assembly 100. Secondary tool 150 may extend, for example, through a space between external sheath 132 and internal sheath 128 or through a lumen defined by one or more walls of external sheath 132 or internal sheath 128. Alternatively, secondary tool 150 may extend through a space between internal sheath 128 and insertion sheath 139.

In FIG. 3F, a snare 147 with a basket 149 coupled to snare 147 are positioned over patch 136 and target tissue 202 contained therein. Snare 147 may be inserted through a lumen of insertion sheath 139 of scope 110. Alternatively, as shown, snare 147 may be inserted into body lumen 200 over the scope or via medical device assembly 100, as described above for secondary tool 150. One or more pull lines 142a, 142b may remain in a tightened configuration to create stability for snare 147, to maintain target tissue 202 within the sack or pouch created by patch 136, to reduce the blood flow to the target tissue, and/or to reduce seeding of the tumor.

As shown in FIG. 3G, snare 147 may be closed or used to cut (e.g., via an electrical current) or separate target tissue 202 from surrounding healthy tissue. Snare 147 may also be used to cut or separate a portion of patch 136 from pull lines 142a, 142b. For example, a portion of patch 136 that is in a center of patch 136 when it is in a flat configuration (FIG. 2) and that is bulging away from a perimeter of patch 136 when it forms a sack or pouch around target tissue 202 may be removed. Basket 149 of snare 147 contains and confines the resected portion of target tissue 202 and the cut portion of patch 136. In alternative configurations, snare 147 may cauterize target tissue 202 to separate it from the surrounding healthy tissue. One or more pull lines 142a, 142b may remain in place and/or be tightened around the base of where target tissue 202 once was, in order to, for example, reduce bleeding or prevent seeding. The target tissue may now be removed by pulling the snare 147 and basket 149 out of body lumen 200 (e.g., by pulling an entirety of scope 110 proximally out of body lumen 200). The user may use a clip or band to secure the closure further (not shown). Additionally or alternatively, another medical device (not shown) may be utilized to cut pull lines 142a, 142b.

In alternative embodiments, patch 136 is positioned over target tissue 202 without the use of medical device 120. In such alternatives, patch 136 may be delivered from insertion sheath 139 of scope 110 by being crimped, folded, bent, or otherwise manipulated to fit to an external surface of insertion sheath 139 of scope 110. In such an embodiment, one or more pull lines (having any properties of pull lines 142a, 142b) may run an entire length of the insertion sheath 139 of scope 110 through a pull line sheath (having any properties of pull line sheath 148) positioned externally on or near insertion sheath 139. The distal ends of the pull lines may be free or may be coupled to one or more actuators, similar to actuator 124 of FIG. 1. In an alternative embodiment, patch 136 may be delivered through a lumen of insertion sheath 139 and the pull lines and/or pull line sheath (having any properties of pull lines 142a, 142b and pull line sheath 148, respectively) may be positioned in a lumen of insertion sheath 139, with a proximal end of the pull lines extending through, for example, a port of handle 101. Alternatively, a user may be able to feed pull line sheath 148 through a working channel of scope 110. This may be accomplished by back loading pull line sheath 148 and one or more pull lines 142 through the working channel of insertion sheath 139. In any of the alternatives described above, to close patch 136, one or more pull lines may be pulled proximally (i.e., towards an operator) through a pull line sheath. This pulls the sides of patch 136 together, and, thus, pulls opposing portions of the healthy tissue surrounding the target tissue together, creating a pouch or sack resembling a polyp, as described above. Similar steps to those above may be taken to resect the target tissue and some or all of patch 136.

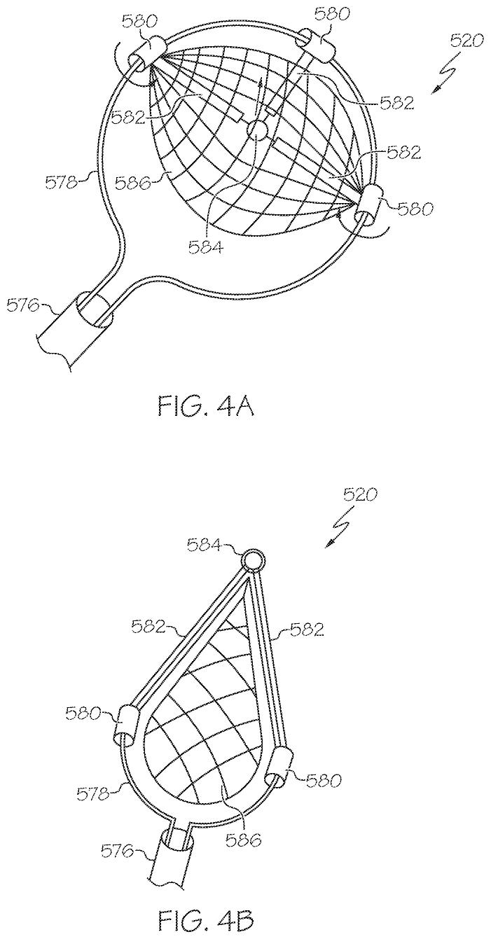

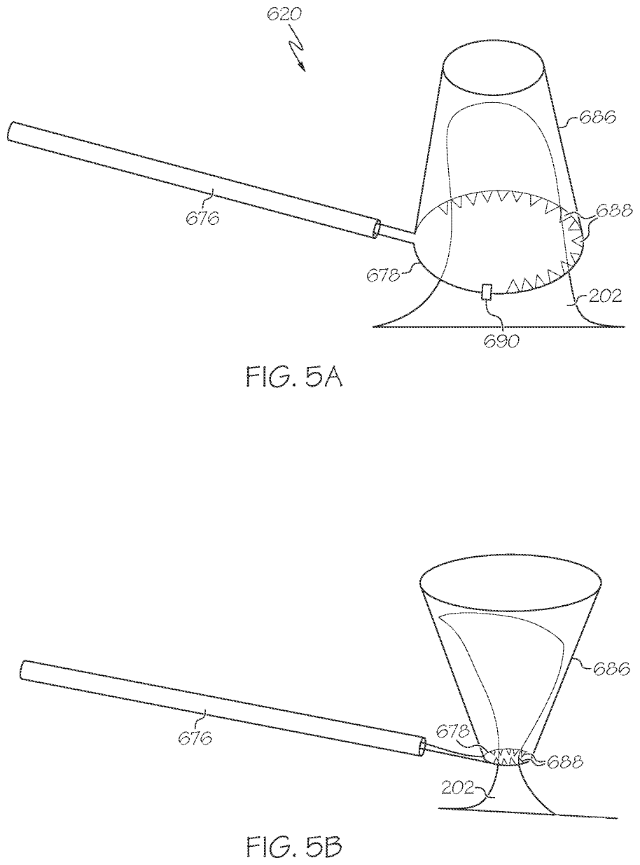

FIGS. 4A and 4B show an alternative medical device 520. FIG. 4A shows medical device 520 in an open configuration. Medical device 520 may comprise a sheath 576 and a wire forming a loop 578 extending from sheath 576. Medical device 520 may be biased to the open configuration shown in FIG. 5A. Loop 578 may be moveable relative to sheath 576. For example, loop 578 may be disposed completely within sheath 576 in a first configuration. In a second configuration, loop 578 may be extended past a distal portion of sheath 576 and expand into the configuration shown.

Dispersed along the length of loop 578 includes two or more tubes 580 (e.g., hypotubes), which may define lumens, through which loop 578 may be received. The two or more tubes 580 are independently moveable relative to loop 578. For example, tubes 580 may rotate relative to loop 578 or may be moveable along a length (e.g., a perimeter/circumference) of loop 578. Tubes 580 may be comprised of one or more biocompatible materials such as stainless steel or one or more plastics (i.e., acrylonitrile butadiene styrene or ABS). A strut 582 extends from each of tube 580 towards a central pivot point 584 positioned near a center of loop 578. The one or more struts 582 may be sufficiently straight and stiff so as to resist bending. A patch 586 extends across a center of loop 578 and may be attached to the one or more struts 582 at various points along the length of the struts 582. Alternatively, patch 586 may be attached to two or more opposing tubes 580. Patch 586 may be comprised of any of the materials previously described with regards to previous embodiments.

FIG. 4B shows medical device 520 in a closed configuration. To achieve such a configuration, the wire forming the loop 578 may be pulled proximally (i.e., towards a user). Additionally or alternatively, loop 578 may be attached to a separate pull wire (not shown) that may be pulled proximally. This movement causes struts 582 to pivot about central pivot point 584 while tubes 580 remain stationary relative to loop 578. Struts 582 and/or patch 586 may comprise barbs or an adhesive material to assist with adhering to the target tissue and pulling the tissue into or against patch 586 as it begins to form a basket when the medical device 520 is transitioned into the closed configuration. While transitioning to or in the closed configuration, as shown in FIG. 4B, struts 582 may bend such that the struts 582 are transverse to a plane defined by the wire forming the loop 578. Struts 582 may point transversely to an axis defined by sheath 576, creating a pyramid or cone shape with patch 586 forming a basket at the center of the pyramid or cone shape.

During use, medical device 520 is used similarly to the previous embodiment. For example, medical device 520 is positioned over a target tissue in an open configuration, as shown in FIG. 4A. Patch 586 may adhere to the target tissue as described above. Once medical device 520 is in position and the target tissue is encompassed by patch 586, the wire forming the loop 578 is pulled proximally (i.e., towards the user). The proximal movement of loop 578 causes loop 578 to shrink, exerting forces on tubes 580, and, in turn, struts 582, causing struts 582 to pivot about central pivot point 584. Tubes 580 may be fixed on loop 578. As struts 582 pivot, patch 586 forms a basket encompassing the target tissue. As the basket is being formed, opposing ends of the tissue surrounding the target tissue are brought together. Similar to the example of FIGS. 1-3G, a polyp-shaped sack or pouch of tissue is formed within the basket formed by patch 586. The polyp-shaped sack or pouch of tissue may now be resected, by, for example, utilizing a secondary tool (not shown) and/or energizing the wire forming the loop 578 or otherwise cutting the tissue with loop 578.

FIGS. 5A and 5B show an alternative medical device 620 in an open configuration (FIG. 5A) and in a closed configuration (FIG. 5B). Medical device 620 comprises a sheath 676 and a wire forming a loop 678, similar to a snare. A serrated surface 688 (i.e., a plurality of teeth) may be positioned on a center-facing portion of loop 678. Serrated surface 688 may face radially inward, towards a center of loop 678, and may be comprised of any suitable material commonly known in the art, including metals or plastics. In some embodiments, serrated surface 688 may not extend around the entirety of loop 678 and/or may be patterned in any suitable period. For example, the serrated surface 688 may be disposed between sections of loop 678 without serrations. Additionally or alternatively, serrated surface 688 may be disposed along a distal portion of loop 678 such that, when loop 678 is pulled proximally into the sheath, serrated surface 688 is not pulled into sheath 676. This may be desirable to avoid damaging sheath 676.

A patch 686 may be coupled at the outermost edges of loop 678 and form a basket. For example, patch 686 may extend away from a plane of loop 678. Patch 686 may be made from any material previously described above with regards to the previous embodiments. Additionally or alternatively, patch 686 may comprise one or more rings of conductive paints (not shown) such that, when electrical energy is supplied to the conductive paints of patch 686, target tissue 202 is resected. The one or more rings of conductive paints (not shown) may be oriented laterally or longitudinally relative to a plane formed by loop 678. Any of the patches described herein with respect to FIGS. 1-5B may include such conductive paints.

In alternatives, medical device 620 may be similar to a bag and may include a string or wire along a rim of patch 686, instead of loop 678. When a thread or wire is pulled, the wire may create a knot so that patch 686 is retained in a closed, sack or pouch like configuration, with tissue retained in the pouch.

Any of the patches described herein (including patch 686) may comprise one or more needles (not shown) on an internal wall (a wall facing the tissue when patch 686 is deployed) of patch 686. The one or more needles may act as a grasper and pierce a target tissue (e.g., target tissue 202) to hold it in place. The one or more needles may be serrated or barbed to sufficiently hold target tissue 202 in place within patch 686.

Medical device 620 may further comprise a locking mechanism 690. Locking mechanism 690 may be similar to those found on a cable tie or zip tie. For example, loop 678 may comprise a notched surface such that, when loop 678 is closed or tightened (as shown in FIG. 5B), locking mechanism 690 can be utilized to hold loop 678 in the tightened configuration. This may be desirable, for example, to hold target tissue 202 in place and within patch 686 during a procedure.

FIG. 5B shows medical device 620 in a closed configuration. In this configuration, using any of the methods described above for actuating medical device 620, the wire forming the loop 678 is pulled proximally, forcing the serrated surface 688 against and/or into target tissue 202, thereby cutting target tissue 202 and/or decreasing a size of loop 678. When target tissue 202 is cut, the resected portion is confined by patch 686 to prevent it from traveling through the body or seeding. As previously stated, locking mechanism 690 may be utilized to hold or lock loop 678 in the tightened or closed configuration. As discussed above with respect to other examples, patch 686, and the resected tissue contained therein, may then be removed from the body.

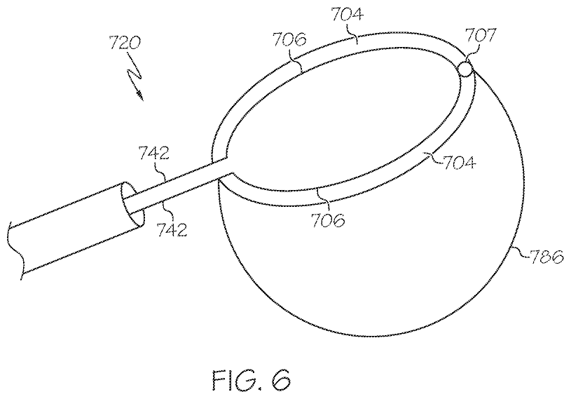

FIG. 6 depicts a medical device 720 having two or more cutting blades 704 and a patch 786 forming a basket. Medical device 720 may have any of the properties of medical device 620, described above. Patch 786 may be coupled to an outer edge of cutting blades 704 in one or more locations. Patch 786 may include or comprise any of the elements and/or characteristics described with regards to the patches or baskets of previous embodiments. For example, an internal surface of patch 786 may include a needle and/or an adhesive surface.

Cutting blades 704 are oriented such that cutting edges 706 of cutting blades 704 face towards each other (i.e., inwards) and toward a tissue to be captured within patch 786. A distal point of each of cutting blades 704 meet at a distal point 707 of medical device 720. One or more lines 742 (i.e., wires) may be coupled to a proximal end of each of cutting blades 704 and extend proximally.

In a first configuration, cutting blades 704 may be slightly curved to form a circle or oval. Cutting blades 704 may be biased in the first configuration. In a second configuration (not shown), lines 742 may be pulled proximally, causing cutting blades 704 to straighten and cutting edges 706 of opposing cutting blades 704 to meet. As cutting blades 704 meet, they may cut tissue and/or capture target tissue within a pouch or sack formed by patch 786 and/or cutting blades 704.

Medical device 720 may be used is a similar manner to the previously described embodiments. For example, medical device 720 may be used to resect a target tissue (not shown), confine or contain the resected tissue within patch 786, and remove the resected tissue from a body lumen.

The patches and/or baskets of the examples depicted in FIGS. 2 and 4A-6C may additionally or alternative include one or more lines (i.e., wires, threads, etc.), surrounding the patch/basket. The one or more lines may be used to cinch (i.e., close) the patch/basket once the target tissue has been resected or cut, thereby confining the resected tissue inside the patch/basket. The one or more lines may be used to form a knot in some embodiments.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device comprising:
a first handle coupled to a first sheath;
a second handle coupled to a second sheath, wherein at least a portion of the first sheath is disposed within a lumen of the second sheath; and
a patch;
wherein, in a first configuration, the first handle and the second handle are separated by a first distance, and the patch is disposed between a distal portion of the first sheath and a distal portion of the second sheath; and
wherein, in a second configuration, the first handle and the second handle are contacting one another or are separated by a second distance, wherein the second distance is smaller than the first distance, and wherein a distalmost end of the second sheath is proximal of a proximal end of the patch.

2. The medical device of claim 1, wherein the first handle comprises a proximal opening configured to receive a distal portion of a handle of an endoscope.

3. The medical device of claim 1, wherein the first handle comprises a feature configured to accommodate a feature of an endoscope.

4. The medical device of claim 1, wherein the patch comprises at least one pull line.

5. The medical device of claim 4, wherein the at least one pull line is arranged within a perimeter of the patch and conforming to a shape of the perimeter of the patch.

6. The medical device of claim 4, wherein each of the at least one pull lines terminates in two free ends extending from a same edge of the patch.

7. The medical device of claim 1, wherein the first handle comprises an actuator configured to move at least one pull line in a proximal direction.

8. The medical device of claim 1, wherein a distal portion of the first sheath includes a balloon coupled to an external surface of the first sheath.

9. The medical device of claim 8, wherein, in the first configuration, the balloon is disposed between the first sheath and the patch. The medical device of claim 8, wherein, in a third configuration, the balloon is inflated and the patch is disposed on the inflated balloon.

11. The medical device of claim 10, wherein, in a fourth configuration, the balloon and the patch are separated.

12. The medical device of claim 1, wherein the patch includes an adhesive on a side of the patch facing away from the first sheath.

13. The medical device of claim 1, wherein the patch is exposed to a liquid via a fluid channel.

14. The medical device of claim 2, wherein the medical device is removably coupled to the endoscope by means of one or more fasteners, adhesives, or a friction fit.

15. The medical device of claim 1, wherein the first sheath comprises a lumen to receive a shaft of an endoscope.

16. A medical device comprising:
a first sheath;
a second sheath, wherein the first sheath is disposed within a lumen of the second sheath;
a balloon; and
a patch,
wherein, in at least one configuration of the medical device, at least a portion of the patch is disposed between the second sheath and at least a portion of the balloon, and at least a portion of the balloon is disposed between at least a portion of the patch and the first sheath.

17. The medical device of claim 16, wherein the patch further comprises at least one pull line arranged within a perimeter of the patch and conforming to a shape of the perimeter of the patch.

18. The medical device of claim 16, wherein the at least one configuration is a first configuration and wherein, in a second configuration, the second sheath is retracted relative to the first sheath and the balloon is inflated.

19. A medical method comprising,
deploying a patch over a lesion within a body lumen;
pulling a pull line of the patch in a proximal direction, such that the patch is surrounds at least a portion of the lesion and a first tissue portion on a first side of the lesion and a second tissue portion on a second side of the lesion are pulled together;
cutting the patch and the lesion contained therein; and
removing the patch and the lesion from the body lumen.

20. The medical method of claim 19, wherein the method further includes:
before deploying the patch, introducing a distal portion of a medical device into a body lumen; and
after introducing the distal portion of the medical device into the body lumen, moving a second handle of the medical device proximally relative to a first handle of the medical device, thereby moving a second sheath of the medical device proximally relative to a first sheath of the medical device and exposing the patch at the distal portion of the medical device.
